(19) United States
(12) Patent Application Publication
(10) Pub. No.: US 2016/0000675 A1
(43) Pub. Date: Jan. 7, 2016

(54) Nail Enamel Composition

(71) Applicants: Dariush Hosseinpour, Edison, NJ (US); Heng Cai, Skillman, NJ (US)

(72) Inventors: Dariush Hosseinpour, Edison, NJ (US); Heng Cai, Skillman, NJ (US)

(73) Assignee: Revlon Consumer Products Corporation, New York, NY (US)

(21) Appl. No.: 14/323,462

(22) Filed: Jul. 3, 2014

(51) Int. Cl. A61K 8/44; A61K 8/35; A61Q 3/02

(52) U.S. Cl. CPC A61K 8/44; A61Q 3/02; A61K 8/355; A61K 2800/81; A61K 2800/592

(57) ABSTRACT

The present invention is directed to a nail enamel composition that includes a photoinitiator, a co-initiator, and an oligomer. The nail enamel composition exhibits increased hardness, toughness, scratch resistance, adhesion, and chip resistance when applied to a natural or synthetic nail.

The properties of nail enamel compositions are generally measured based on their hardness, toughness, scratch resistance, adhesion, and chip resistance. Conventional nail enamel compositions generally contain a film forming component, a plasticizer component, a solvent component, and optionally, a pigment component. These conventional nail enamels generally exhibit average levels of hardness, toughness, scratch resistance, adhesion, and chip resistance.

It is desirable that a nail enamel possess increased hardness, toughness, scratch resistance, adhesion, and chip resistance to provide longer wear and to decrease the frequency of reapplication necessary. Such desire and need is met by the present nail enamel composition.

### SUMMARY OF THE INVENTION

The present invention is directed to a nail enamel composition comprising a combination of a photoinitiator, an oligomer and an optional co-initiator and said composition dries ≦5 minutes when applied to a natural or synthetic nail exposed to a visible light at a wavelength of about 380 nm to about 700 nm. The light can come from a source such as natural daylight or a visible light lamp. The composition exhibits better wear, hardness, scratch resistance, adhesion, and chip resistance.

In another embodiment, the oligomer is a urethane acrylate oligomer. The oligomer may be present in the nail enamel composition in an amount of up to about 25% w/w. The oligomer has a molecular weight greater than 500 g/mole and a functionality of about >1 and <18. In a preferred embodiment the oligomer functionality is about 6.

In one embodiment, the photoinitiator is camphorquinone. The photoinitiator may be present in the nail enamel composition in an amount of up to about 10% w/w of the oligomer. In an embodiment the photoinitiator,is a type one, a type two, a uv photoinitiator, or a combination of photoinitiator types.

In another embodiment, the co-initiator is an amine co-initiator.

The nail enamel composition of the present invention may further comprise at least one of: a film forming component, a solvent component, a plasticizer component, a suspending agent, and a pigment component.

The present invention is also directed to a method of coating a natural or a synthetic nail which includes the steps of applying (lay or spread on) the nail enamel composition of the present invention to the natural nail or synthetic nail and exposing the natural or synthetic nail with the nail enamel applied to a wavelength of 380-700 nm for ≦5 minutes.

In an embodiment the composition contains an optional monomer.

### DESCRIPTION OF THE DRAWINGS

FIG. 1 Graphs hardness as a function of exposure to visible light for compositions of Example

### DETAILED DESCRIPTION OF THE INVENTION

The present invention is directed to a nail enamel composition comprising a photoinitiator, an optional co-initiator, and an oligomer. The nail enamel composition exhibits an increased hardness, and increased adhesion, a decreased abrasion, an increased chip resistance, and increased flexibility, and increased fracture toughness when compared to conventional nail enamel compositions.

The oligomer used in the nail enamel composition of the present invention has a molecular weight of greater than 500 g/mole, preferably, in a range of 2,000-4,000 g/mole, and more preferably less than 25,000 g/mole.

The oligomer may have a structure that is linear, branched, or hyper-branched. Further, the oligomer may have one or more type of backbone chemistry; examples of the backbone chemistry include, but are not limited to, urethane, epoxy, polyester, silicone, and acrylic. The oligomer may also have one or more functional groups; examples of the functional group include, but are not limited to, acrylate and methacrylate.

The oligomer has a functionality that is about ≧1 and about ≦18. Functionality is determined by the number of acrylate or methacrylate groups on the oligimer. Oligimers with different functionalities can be mixed and used in combination.

The most preferred result is obtained using an oligomer having a functionality of 6. The most preferred result is one where the nail enamel dries faster, lasts longer on the nail, and is resistant to chipping and fading when compared to another nail enamel without the oligomer in the formula.

The oligomer may be present in the nail enamel composition of the present invention in an amount of up to about 25% w/w of the composition.

The photoinitiator used in the nail enamel composition of the present invention may be a type I (cleavage mechanism) photoinitiator or a type II (H-abstraction mechanism) photoinitiator. Examples of a type I photoinitiator include, but are not limited to, acylphosphine oxides and benzoin. The photoinitiator types may be used alone or in combination with uv photoiniators. Examples of a type II photoinitiator include, but are not limited to, camphorquinone, benzophenone and its derivatives, and titanocenes. In addition, the photoinitiator used in the nail enamel composition of the present invention has an absorption band in the visible light region. The visible light region used herein is about 380 nm to about 700 nm. Preferably, the photoinitiator used in the nail enamel composition of the present invention is a type II (H-abstraction mechanism) photoinitiator. More preferably, the photoinitiator is camphorquinone.

The photoinitiator may be present in the nail enamel composition of the present invention in an amount of up to about 10% w/w of the oligomer.

The co-initiator used in the nail enamel composition of the present invention is a hydrogen-donor molecule. The presence of the co-initiator overcomes the surface tackiness. The curing rate and degree of crosslinking depend on the choice of co-initiator. Examples of coinitiators include, but are not limited to, amines; such as Ethyl 4-dimethylaminobenzoate from Hampford Research Inc and Triethanolamine from Dow Chemical; acrylated amines such as, CN373 from Sartomer Company; amine-modified acrylate oligomer; cyclic acetals, such as, 1,3-benzodioxole and its derivatives such as 2-hexyl-1,3-benzodioxole; and silanes. Tertiary amines have been used with particular success.

The nail enamel composition of the present invention may also include monomers.

An exemplary reaction scheme of the photopolymerization is shown below. In the exemplary reaction scheme, camphorquinone photoinitiator (CQ) and an amine co-initiator are shown; however, it is understood that the present invention is not limited to such embodiment.

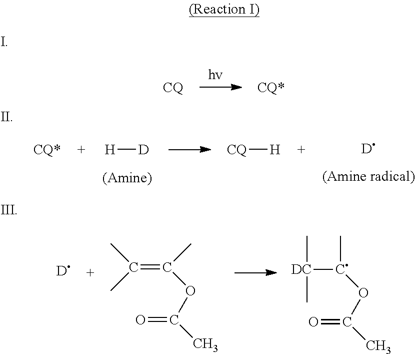

As seen above, camphorquinone initiates the free radical polymerization under visible light. The free radical polymerization is accelerated by the presence of the amine co-initiator. CQ absorbs light due its transition of dicarbonyl group (Stage I). The excited CQ abstracts a hydrogen atom from the co-initiator (H-donor molecule (H-D)) and produces a primary radical, e.g., amine radical (Stage II), which attacks the carbon-carbon double bonds of oligomers or monomers (Stage Ill) thus generating primary propagating radicals to attack other oligomers or monomers and initiate polymerization. The resulting polymers contribute to the increased wear, hardness, toughness, scratch resistance, adhesion, and chip resistance characteristics of the nail enamel.

The nail enamel composition of the present invention may further comprise at least one of: a film forming component, a solvent component, a plasticizer component, and a pigment component.

A film-forming component is an optional, but preferred, component of the nail enamel compositions of the present invention. If used, the film-forming component is present in an amount sufficient to provide a stable film on the nail following the application of the nail enamel composition to the nail, but not so high of a concentration that the nail enamel composition is unable to flow freely onto a brush and from the brush onto the nail.

Examples of a film forming component include, but are not limited to, nitrocellulose, cellulose acetate butyrate, polyurethanes, mixtures of polyurethanes with cellulose acetate butyrate or with nitrocellulose, and other suitable film forming agent known in the art. The film-forming component may further comprise acrylics, acrylates, polyurethanes, vinyls, acrylonitrile/butadiene copolymers, styrene/butadiene copolymers, epoxies, and any other polymer or copolymer capable of adaptation to a nail enamel system, such as the copolymers disclosed in U.S. Pat. No. 4,762,703, the disclosure of which is hereby incorporated by reference.

The film-forming component may be present in the nail enamel composition in an amount of about 10% w/w to about 40% w/w of the nail enamel composition.

A solvent component is an optional, but preferred, component of the nail enamel compositions of the present invention. If used, the solvent component should be inert to the user's nail and to the other components of the nail enamel composition, should be capable of dissolving or dispersing the other components of the nail enamel allowing the components to flow onto the nail, and should be able to evaporate from the nail in a matter of minutes at room temperature and pressure.

Examples of solvents include, but are not limited to, isopropanol, butyl acetate, ethyl acetate, glycol ethers, alkyl lactates, mixtures thereof, and other suitable solvents known in the art.

A suitable amount of the solvent component will generally lie in the range of about 40% w/w to about 60% w/w of the composition.

A plasticizer component is an optional, but preferred, component of the nail enamel compositions of the present invention. If used, the plasticizer component should improve flexibility and resistance of the nail enamel, for example, to soap and water.

Examples of a plasticizer component include, but are not limited to, alkylaryl or cycloalkyl phthalate; camphor; a polymeric component formed by condensation polymerization of formaldehyde or other aldehyde, typically an aromatic sulfonamide-aldehyde condensation resin and other suitable plasticizer known in the art. Other examples of a plasticizer component include glyceryl tribenzoate and other plasticizers disclosed in U.S. Pat. No. 5,066,484, the disclosure of which is hereby incorporated by reference.

A suspending agent is an optional component of the nail enamel compositions of the present invention. If used, the suspending agent should help suspend the pigments in the nail enamel, and helps adjust the viscosity to achieve desired flowability. Examples of a suspending agent include montmorillonite clays, and treated clays such as stearalkonium hectorite. The amount of the suspending agent used depends on the desired flow characteristics of the nail enamel, but an amount of about 0.5% w/w to about 2% w/w of the nail enamel composition are generally satisfactory.

The nail enamel composition of the present invention may be clear, i.e. unpigmented, or may include a pigment component. Suitable pigments include all inorganic and organic pigments which are usable in cosmetic formulations. Particular examples include carmine, bismuth oxychloride, zinc oxide, ferric oxide, ferrous oxide, kaolin, ultramarine violet, ultramarine blue, chromium oxide, chromium hydroxide, silica, and manganese violet. Other examples include lakes of organic colorants such as D&C Red No. 7 Calcium Lake, FD&C Yellow No. 5 Aluminum and Zirconium Lakes, D&C Red No. 6 and No. 9 Barium Lakes, D&C Red #34 Calcium Lake, and D&C Red No. 30. Additional examples include talc, mica, titanium dioxide; any of the foregoing carried on the surface of talc, mica or titanium dioxide; and titanated mica.

The term “pigment” includes mixtures of two or more of the above-mentioned pigments, and includes any of the above-mentioned pigments whose surfaces have been treated by the addition of silicone, lecithin, or other surface treatments.

The amounts of any particular ingredients comprising the pigment component will depend on the shade desired. In general, the pigment component comprises about 0.01% w/w to about 10% w/w of the nail enamel composition.

The nail enamel composition of the present invention may be manufactured by thoroughly mixing together all the components. Examples of satisfactory equipment and how to use it are readily apparent to one of ordinary skill in this art.

If a plasticizer component and a film-forming component are present, the preferred mixing procedure is first to mix the plasticizer component into a solution or to mix it into a solution to which only the film-forming component has already been added, and thereafter to mix in the other ingredients of the nail enamel. This procedure is preferred also whenever the plasticizer is relatively difficult to solubilize.

The nail enamel composition described above is applied to a natural or synthetic nail and exposed to a light source that supplies a wavelength of about 380 nm to about 700 nm. The applied nail enamel dries to the touch in ≦to about 5 minutes after exposure to the light source. A nail is exposed when after application of the nail composition the hand or foot is left unmoved under a light source until the nail enamel is hard to the touch and forms a solid coating on the natural or synthetic nail.

The light source is either natural daylight or a visible light lamp. Examples of light sources include, but are not limited to, fluorescent, incandescent, LED, or halogen lamps. The output of light source could be between 2 W to 500W. Preferably, it should be between 5 to 220W. The color temperature associated with the light source could be from 2500 to 7500K. Examples of Lamp manufacturers are Philips, General Electric, Lighting Science, Ledtronics. The light wavelength range associated with the lamps is 380 nm to 700 nm.

As used herein, “about” shall generally mean within 20 percent, preferably within 10 percent, and more preferably within 5 percent of a given value or range.

When referencing a natural or synthetic nail the intended meaning for a natural nail is the keratinous material that grows from fingers and toes on a mammal. A synthetic nail is any material that is applied to the natural nail with the intent that the synthetic nail acts like a natural nail. Examples of synthetic nails, include but are not limited to, silk, plastic, and a polymer mix.

Various tests may be carried out to compare the nail enamel composition of the present invention containing a photoinitiator, an optional co-initiator, and an oligomer, with a composition without these components.

The various tests are well-known in the art. Certain examples of the tests are provided below:

| Property | Test(s) |
|---|---|
| Hardness | Pendulum hardness test |
| | http://www.sheeninstruments.com/datasheets/pen2.pdf) |
| | Buchholz indentation test |
| | (http://www.gardco.com/pages/hardness/buchholz |
| | BH2000.cfm |
| | Dur-O-Test, pencil hardness test, |
| | http://www.byk.com/en/instruments/physical- |
| | properties/hardness/pencil-hardness-tester.html |
| | Mohs hardness test |
| | http://www.gordonengland.co.uk/hardness/mohs.htm |
| | Vickers hardness test, |
| | http://www.qualitest-inc.com/vickers-hardness-testers.htm |
| | Gardner/Sward hardness test |
| | http://www.gardco.com/pages/hardness/swardrocker.cfm |
| Adhesion | Cross-cut test (for dry sample); for wet adhesion, the coated |
| | sample would be submerged in an aqueous solution for a |
| | certain time, then a cross-cut test would be performed |
| Abrasion | Abrasion-scrub test and Taber abrasion test |
| Chip | Standard test method for chipping resistance of coatings |
| resistance | |
| Flexibility | Conical mandrel test and cylindrical mandrel test |
| Fracture | ASTM 5045: Standard test method for fracture |
| toughness | toughness measurement; and tests as described |
| | in the following articles: |
| | Szekrenyes, A., “Overview on the Experimental |
| | Investigations of the Fracture Toughness in |
| | Composite Materials.” available at |
| | http://hej.sze.hu/MET/MET-020507-A/met020507a.pdf |
| | Zhang, Y., et al., “Mode I and II Fracture Behavior of |
| | Adhesively-Bonded Pultruded Composite Joints,” |
| | Engineering Fracture Mechanics, 77, pp. 128-143, 2010 |
| | Fasce, L., et al., “Evaluation of Impact Fracture |
| | toughness of Polymeric Materials by Means of the |
| | J-Integral Approach,” Polymer Engineering and |
| | Science, vol. 43, no. 5, pp. 1081-95, May 2003 |
| | Hashemi, S., et al., “Corrections Needed in Double- |
| | Cantilever Beam Tests for Assessing the Interlaminar |
| | Failure of Fibre-Composites,” Journal of Material |
| | Science Letters, 8, pp. 125-129, 1989 |

The examples below illustrate exemplary formulations of the nail enamel composition according to the present invention. It is not intended as a limitation upon the scope of the present invention.

### EXAMPLE 1

Different nail coating compositions containing different amounts of light-curing urethane acrylate oligomer were prepared according to the following table.

The films of compositions were applied at a thickness of 6 mils, and were left in the dark for 2 hours.

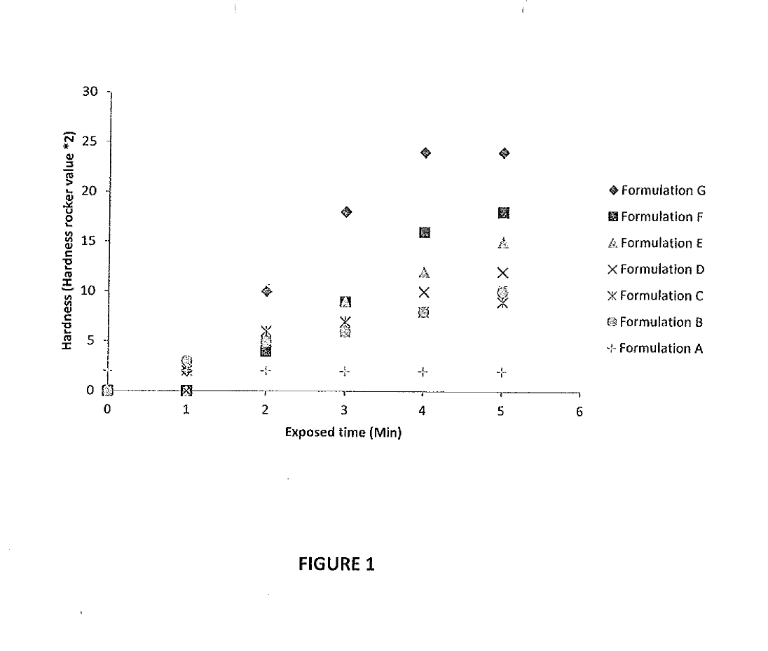

The samples were then subjected to the visible light (Fluorescent lamp 100W with a 6500K color temperature) and the hardness was measured after every minute of exposure. Sward hardness test (Gardner/Sward Hardness Rocker Model# GS1) was used for hardness measurement. The results are found in FIG. 1.

| Formula | A | B | C | D | E | F | G | H |
|---|---|---|---|---|---|---|---|---|
| Butyl acetate | 20.49 | 18.441 | 15.3675 | 10.245 | 5.1225 | 2.049 | 0 | 15.3675 |
| Propyl acetate | 5.8 | 5.22 | 4.35 | 2.9 | 1.45 | 0.58 | 0 | 4.35 |
| Ethyl acetate | 34.7 | 33.355 | 31.3375 | 27.975 | 24.6125 | 22.595 | 21.25 | 31.3375 |
| Acetone | 0 | 2.125 | 5.3125 | 10.625 | 15.9375 | 19.125 | 21.25 | 5.0575 |
| IPA | 7.17 | 6.453 | 5.3775 | 3.585 | 1.7925 | 0.717 | 0 | 5.3775 |
| Acetyl tributyl citrate | 3.8 | 3.42 | 2.85 | 1.9 | 0.95 | 0.38 | 0 | 2.85 |
| Triacetin | 0.9 | 0.81 | 0.675 | 0.45 | 0.225 | 0.09 | 0 | 0.675 |
| Nitrocellulose | 14.58 | 13.122 | 10.935 | 7.29 | 3.645 | 1.458 | 0 | 10.935 |
| Tribenzoin | 10.9 | 9.81 | 8.175 | 5.45 | 2.725 | 1.09 | 0 | 8.175 |
| Tetrabutyl phenyl | 0.2 | 0.18 | 0.15 | 0.1 | 0.05 | 0.02 | 0 | 0.15 |
| hydroxybenzoate | | | | | | | | |
| Stearalkonium Bentonite | 1.4 | 1.26 | 1.05 | 0.7 | 0.35 | 0.14 | 0 | 1.05 |
| Citric acid | 0.0125 | 0.01125 | 0.009375 | 0.00625 | 0.003125 | 0.00125 | 0 | 0.009375 |
| Malic add | 0.0375 | 0.03375 | 0.028125 | 0.01875 | 0.009375 | 0.00375 | 0 | 0.028125 |
| Serica (Silk Powder) | 0.01 | 0.009 | 0.0075 | 0.005 | 0.0025 | 0.001 | 0 | 0.0075 |
| Urethane acrylate | 0 | 5 | 12.5 | 25 | 37.5 | 45 | 50 | 12.5 |
| oligomer | | | | | | | | |
| Camphorquinone | 0 | 0.25 | 0.625 | 1.25 | 1.875 | 2.25 | 2.5 | 0.625 |
| Ethyl 4- | 0 | 0.5 | 1.25 | 2.5 | 3.75 | 4.5 | 5 | 1.25 |
| Dimethylaminobenzoate | | | | | | | | |
| Pigment Red 7 Lake | 0 | 0 | 0 | 0 | 0 | 0 | 0 | 0.003 |
| Pigment Yellow 5 Lake | 0 | 0 | 0 | 0 | 0 | 0 | 0 | 0.002 |
| Titanium dioxide | 0 | 0 | 0 | 0 | 0 | 0 | 0 | 0.25 |
| Sum | 100 | 100 | 100 | 100 | 100 | 100 | 100 | 100 |

What is claimed is:

1. A nail enamel composition comprising a combination of a photoinitiator and an oligomer, wherein the oligomer has a molecular weight greater than 500 g/mole and a functionality of about >1 and <18, and said composition dries ≦5 minutes when applied to a natural or synthetic nail exposed to a visible light at a wavelength of about 380 nm to about 700 nm.

2. A nail enamel composition of claim 1, optionally containing a co-initiator.

3. The nail enamel composition of claim 1, wherein the oligomer is present in an amount of up to about 25% w/w.

4. The nail enamel composition of claim 1, wherein the photoinitiator is present in an amount of up to about 10% w/w of the oligomer.

5. The nail enamel composition of claim 4, wherein the photoinitiator is camphorquinone.

6. The nail enamel composition of claim 4, wherein the oligomer is a urethane acrylate oligomer.

7. The nail enamel composition of claim 1, wherein the oligomer has a functionality of about 6.

8. The nail enamel composition of claim 1, wherein the photoinitiator is a type one, type two, UV or combination of photoinitiator types.

9. The nail enamel composition of claim 2, wherein the co-initiator is an amine co-initiator.

10. The nail enamel composition of claim 1, further comprising at least one of; a film forming component, a solvent component, a plasticizer component, a suspending agent, and a pigment component.

11. A method of coating a human nail comprising:
a. applying the composition of claim 1 to a natural or synthetic nail; and
b. exposing the natural or synthetic nail with the nail enamel applied to a wavelength of 380-700 nm for ≦5 minutes.

12. The composition of claim 1 wherein the composition, exhibits an increased hardness, and increased adhesion, a decreased abrasion, an increased chip resistance, and increased flexibility, and an increased fracture toughness.

13. The composition of claim 1, wherein the composition contains an optional monomer.

14. A nail enamel composition prepared by a method of reaction I.

15. The composition of claim 1, wherein the light is a natural daylight.

16. The composition of claim 1, wherein the light is a visible light lamp.

17. A composition comprising a combination of a photoinitiator and an oligomer prepared a process of Reaction I.

18. The composition of claim17, wherein the composition is a nail enamel.

19. The composition of claim 17, wherein the composition optionally contains a co-initiator.

20. The composition of claim 17, wherein the photoinitiator is camphorquinone.
